(19) United States
(12) Patent Application Publication
(10) Pub. No.: US 2022/0233389 A1
(43) Pub. Date: Jul. 28, 2022

(54) PORTABLE ROBOTISED APPARATUS WITH FUNCTIONAL ELECTROSTIMULATION FOR ASSISTED JOINT REHABILITATION

(71) Applicant: GOGOA MOBILITY ROBOTS, SL., URRETXU (Guipuzcoa) (ES)

(72) Inventors: Carlos FERNANDEZ ISOIRD, URRETXU (Guipuzcoa) (ES); Juan Antonio MARTIN PRADO, URRETXU (Guipuzcoa) (ES); Elene SANTOS ZALACAIN, URRETXU (Guipuzcoa) (ES); lker MARINELARENA ARRIZABALAGA, URRETXU (Guipuzcoa) (ES)

(21) Appl. No.: 17/609,491

(22) PCT Filed: Jun. 8, 2020
(86) PCT No.: PCT/ES2020/070376

§ 371 Date: Nov. 8, 2021

(30) Foreign Application Priority Data
Jun. 19, 2019 (ES) P201930557

(51) Int. Cl. A61H 3/00; A61H 1/02; A61N 1/36

(52) U.S. Cl. CPC A61H 3/00; A61H 2201/5097; A61N 1/36003; A61N 1/36034; A61H 2201/0157; A61H 2201/1659; A61H 2201/10; A61H 2201/165; A61H 2003/007; A61H 2201/5064; A61H 2201/5058; A61H 2201/5061; A61H 2201/1207; A61H 2201/1463; A61H 2201/5007; A61H 1/024

(57) ABSTRACT

A portable robotised apparatus with functional electrostimulation is provided having an auxiliary actuator for the limb joint to be rehabilitated; an orthosis for attaching to the patient; sensors for measuring parameters; and an autonomous power source. The portable robotized apparatus is characterised in that it also has: a 16-channel electric-pulse generator; a non-invasive multielectrode array; control electronics, to coordinate the actuator and the electric pulse generator, collect the measurement from the sensors and carry out real-time control; and a control algorithm to command, inter alia, the electric-pulse generator. The actuator operates in a coordinated manner in real time with the electric-pulse generator which is applied to the affected limb by means of the electrode array.

### OBJECT OF THE INVENTION

The object of the invention relates to a portable robotised apparatus with a degree of freedom, operated by an electromechanical system and integrated with a system of electrostimulation by a hybrid control.

The system which is object of the invention assists the patient in an intelligent manner by a control that coordinates movement by the action of the robot system and electrostimulation of the affected muscles.

Generally used for assisted functional rehabilitation of joints, and more specifically for the rehabilitation of the knee, it can be used during the patient's natural gait.

### BACKGROUND OF THE INVENTION

In the current state of the art the so-called Robotic Assisted Rehabilitation Systems (also known as RAR) are already known that are used in the clinical rehabilitation of patients with different types of injuries, whether these are the result of accidents or of acquired brain injury. Robotic Assisted Rehabilitation Systems (RAR) are emerging field in the clinical rehabilitation of patients with different types of injuries, either as a result of traumatic accidents, or as a result of Acquired Brain Injury (ABI), due, for example, to ictus or to cerebrovascular accidents. They are also an innovative area of research applied to the understanding of neuromuscular injuries and their rehabilitation by the use of robotised devices.

In the current state of the art, Functional Electrical Stimulation is also known (known as FES), used for several decades as a rehabilitation technique to recover, totally or partially, loss of motor functionality in disabled subjects.

### TECHNICAL PROBLEM TO BE RESOLVED

Disorders of the knee joint are frequent, as a result of weakness of the quadriceps, patellofemoral pain syndrome, or injuries, stroke, post-polio syndrome, multiple sclerosis or damage in the spinal cord. The known devices for knee rehabilitation benefit some or all of these patients. Nevertheless, with few exceptions, existing orthopaedic options are limited to passive technologies that cannot provide the necessary assistance to replicate the function of a healthy knee.

A step forward in rehabilitation tools is the use of portable robotic systems (portable RAR) that are joined to the affected leg and make it possible to supplement the effort made by the patient, helping them to make a movement close to the natural pattern of walking and promoting the rehabilitation process. For example, and inter alia, in document ES2689218 of the applicant a robotised system for assisted functional rehabilitation of joints (RAR) is described that integrates drive (normally electric actuators), mechanical transmission, electronics, sensors and control algorithms. For example, and inter alia, in document ES1182486 of the applicant, a mechanical transmission assembly is described for this type of rehabilitation equipment.

Functional Electrical Stimulation (FES) uses electrical stimuli in peripheral nerves to produce involuntary contractions to obtain a functional movement. The use of Functional Electrical Stimulation (FES) for assistance in walking offers numerous advantages for patients with motor disability: it prevents muscular atrophy, reduces spasticity [as described by T. W. J. Janssen, R. Glaser, D. B. M. Shuster, Clinical efficacy of electrical stimulation exercise training: effects on health, fitness, and function, Topics in Spinal Cord Injury Rehabilitation 3 (3).] It improves vascular circulation and it has been shown to provide psychological benefits [as described by M. B. Popovic, D. B. Popovic, T. Sinkjaer, A. Stefanovic, L. Schwirtlich, Clinical evaluation of functional electrical therapy in acute hemiplegic subjects, Journal of Rehabilitation Research & Development 40, 5].

Nevertheless, the individual use of these types of techniques (RAR) and (FES) described offers numerous disadvantages. Unresolved problems are, inter alia, the early appearance of muscular fatigue and the development of erratic movement with great accelerations due to the difficulty of controlling the joint torque.

### DESCRIPTION OF THE INVENTION

The applicant has verified that when the rehabilitation tools of the robotic systems for assisted functional rehabilitation are integrated with a system of electrical muscular stimulation the recovery process accelerates.

The object of the invention integrates in the equipment/apparatus a robotic-assisted rehabilitation (RAR) system and a functional electrical stimulation (FES) system so that the combined action of both systems makes it possible to accelerate and improve the rehabilitation process, eliminating the disadvantages of the individual application of electro-stimulation. Part of a robotised system for assisted functional rehabilitation of joints (previous development of the applicant) and of a system of functional electrical stimulation (FES) and develops an algorithm that simultaneously coordinates the assistance offered by the robotised system (strength, speed and position) and the matrix of electrodes for functional electrical stimulation to induce the contraction-extension of the muscles involved in each movement of the knee in real time. It is characterized in that it consists of:
a 16-channel electric-pulse generator;
a non-invasive multielectrode array;
control electronics to coordinate the actuator and the electric-pulse generator, collect the measurement of the sensors and carry out real-time control; and
a control algorithm to command, inter alia, the electric-pulse generator.

Applicable in general for assisted functional rehabilitation of joints, and more specifically for the rehabilitation of the knee, this invention can be used during the natural gait of the patient and causes the rehabilitation process to be more efficient and effective, activating the muscles and generating a controlled gait, thus obtaining better rehabilitation is less time.

Other configurations and advantages of the invention can be deduced from the following description, and from the subsidiary claims.

### DESCRIPTION OF THE DRAWINGS

To better understand the object of the invention, a preferential form of embodiment is represented in the attached figures, subject to accessory changes that due not essentially alter it. In this case:

FIG. 1 represents the architecture of the robotised apparatus: a general plan view with its basic integrating components and the interconnection between them.

FIG. 2 represents the robotised apparatus that is the object of the invention, assembled and ready for use in an extended position.

FIG. 3 represents a general view of the apparatus with its components arranged for assembly.

### DETAILED DESCRIPTION OF A PREFERENTIAL EMBODIMENT

Described below is an example of practical, non-limiting, embodiment of this invention. Other modes of embodiment in which accessory changes are introduced that do not essentially alter it are in no way ruled out

The object of the invention is a portable robotised apparatus with functional electrostimulation for assisted rehabilitation of joints, characterized in that it consists of a Robotic Assisted

Rehabilitation (RAR) system portable by the patient during the natural gait, an integrated functional electrical stimulation (FES) system, the assembly being portable.

The apparatus which is the object of the invention assists the patient in an intelligent manner by a control that coordinates movement (of mechanical transmission) and the electro-stimulation of the affected muscles. It is comprised of at least:
An electrical actuator (1), which consists of a motor (11) and a mechanical transmission (12). The motor (11) is a brushless electric motor and actuates the mechanical transmission (12) thus making possible the revolving of the arms (13) carrying the orthosis (2) and auxiliaries of the corresponding joint of the limb to be rehabilitated;
Orthosis (2) for attaching of the device to the patient;
Control electronics (4)
Sensors (3) of several types:
   Hall effect sensors for regulation of the motor (11);
   absolute magnetic encoder for the control of the position; and
   strain gauges to measure the force of interaction between patient and device;
A power source (6);
An electro-stimulation unit (7) that uses a 16-channel electro-pulse generator (9);
A non-invasive superficial multielectrode array (8);
Control software, and
Protections/finishes (10) to improve final appearance.

The following form part of the apparatus:
the auxiliary actuator (1), of the corresponding joint of the limb to be rehabilitated.:
the orthosis (2), for attachment to the patient;
the sensors (3) for measuring both the parameters and the angular position, speed, strength and interaction torque between the system and the limb to be rehabilitated; and
the power source (6) supplying the energy necessary for the operation of the unit in portable mode.

The following is the object of the invention:
the 16-channel electric-pulse generator (9);
non-invasive multielectrode array (8);
the control electronics (4), to coordinate the actuator (1) and the electric-pulse generator (9) and carrying out real-time control; and
the control algorithm (5) to command the electric-impulse generator (9).

The use of protectors/finishes (10) to improve the final appearance is accessory.

The object of the invention is also characterized in that the actuator (1) is a mechanical transmission that includes, at least, a motor (11) and a reducer (12) with a transmission ratio of 1:160.

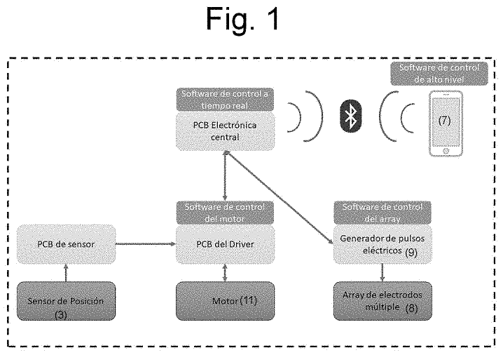

On the one hand, as shown in FIG. 1, the multielectrode array (8) is controlled by the electric-pulse generator (9). The electric-pulse generator (9) contains embedded software that controls the discharges (the synchronization, the intensity, the frequency, etc.) and that, at the same time, can communicate bidirectionally with the software embedded in the electronic card called “Central Electronic PCB” in FIG. 1. At the moment that the control algorithm of electronic card “Central Electronic PCB” sends the order to the stimulator, it receives the order and begins to generate the stimuli to apply with the multielectrode array (8).

On the other hand, the motor (11) used in the robotic system has the electronic card, called “Driver PCB” in FIG. 1, which contains the algorithm to control the due operation of the actuator (1). This due operation consists of sending the correct activation pattern to the poles of the motor (11) so that they can cause a suitable rotation in the rotary shaft.

“Central Electronic PCB” card supervises and coordinates in real time both the electric-pulse generator (9) and the robotic rehabilitation system (through the electronic card “Driver PCB”), so that the operation of both systems is synchronized by the appropriate control algorithm.

This control algorithm is implemented by software integrated into the card, the “Central Electronic PCB” card and that in FIG. 1 has been identified/called “Real-time control software”, which can manage the data received by the position sensor (3) through an electronic board called “Sensor PCB” in FIG. 1 to control the motor (11) using the software of the electronic called “Driver PCB” as a bridge.

During the management, the robotic system receives commands by Bluetooth from the high-level control software and sends information about the status of the system. The result is a robotic system that is commanded by the user by, for example, a mobile phone application that rehabilitates the joint by the repetition of the movements made by the motor (11), coordinating these movements with the electric stimulation of the muscles that intervene in the movement.

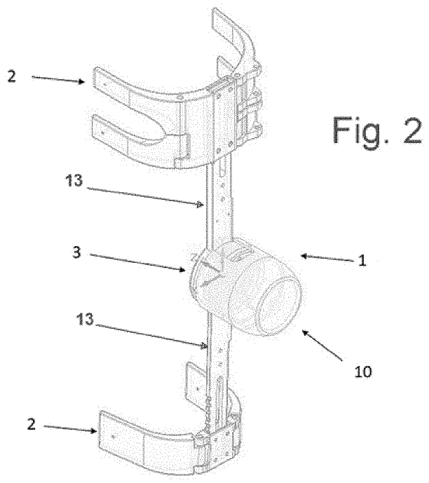

As can be seen in FIG. 2 which represents the assembled device, the motor (11) and the reducer (12) are covered by a plastic case (10) and the electrode array (8) is under the orthosis (2) of the thigh.

For the correct operation of the apparatus, the orthoses must be correctly fastened (2), the desired pattern of movement must be chosen on the electro-stimulation unit (7), and the rehabilitation session must begin.

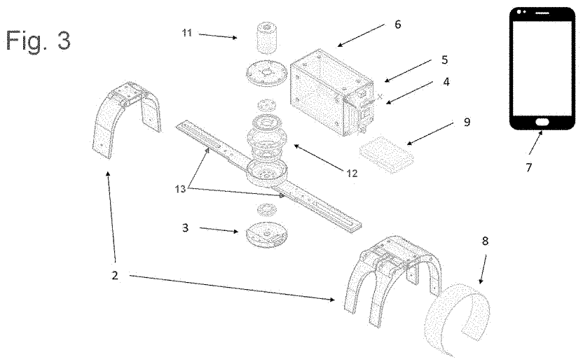

The electro-stimulation unit (7) can be, for example, a BoneSTIM® stimulator or a mobile application. See FIG. 3.

The materials, dimensions, proportions and in general, those other accessory or secondary details that do not essentially alter, change or modify the proposal can be variable.

The terms in which this report is written are a true and accurate reflection of the object described, and must be taken in their broadest sense and never in a limiting manner.

1. A portable robotised apparatus with functional electro-stimulation for assisted rehabilitation of joints, integrating in the same apparatus a Robotic Assisted Rehabilitation (RAR) system portable by the patient during normal gait, a system of functional electrical stimulation (FES), the unit being portable, said apparatus consists of an auxiliary actuator of the corresponding limb joint to be rehabilitated; orthosis for attaching to the patient; sensors that can measure both the parameters and the angular position, speed, force and interaction torque between the system and the limb to be rehabilitated; and a power source supplying the energy necessary for the operation of the unit, the portable robotised apparatus comprising:
a) a 16-channel electric-pulse generator;
b) a non-invasive multielectrode array;
c) control electronics, to coordinate the actuator and the electric-pulse generator, collect the measurement from the sensors and carry out real-time control;
d) control algorithm to command, inter alia, the electric pulse generator, said algorithm coordinates simultaneously the assistance offered by the robotised system (force, speed and position) and a matrix of electrodes for functional electro-stimulation to induce the contraction-extension of the muscles involved in each movement of the knee in real time.

2. The robotised apparatus, according to claim 1, wherein the actuator functions in a coordinated manner in real time with the electric pulse generator that is applied to the affected limb by the electrode array.

3. The robotised apparatus, according to claim 1, wherein the actuator is a mechanical transmission that includes at least one motor and one reducer with a transmission ratio of 1:160.

4. The robotised apparatus, according to claim 1, wherein the control electronics includes, at least one main control board “Central Electronic PCB ”, a board for control of the joint “Driver PCB”, a “PCB del sensor” board to collect the measurement of the position sensors and another board for the connection between the robotic system and the electric-pulse generator.

5. The robotised apparatus, according to claim 2, comprising a multiple and configurable electrode array.

6. The robotised apparatus, according to claim 4, wherein the electric-pulse generator contains embedded software that controls the discharges (synchronization, intensity, frequency) and that at the same time, can communicate bidirectionally with the software embedded in the electronic card called “Central Electronic PCB”.

7. The robotised apparatus, according to claim 1, wherein the robotic system receives commands by Bluetooth from the high-level control software and sends information about the status of the system, is commanded by the user with a mobile telephone application that rehabilitates the joint through the repetition of the movements made by the motor, coordinating these movements with the electrical stimulation of the muscles that intervene in the movement.
